(19) United States
(12) Patent Application Publication
(10) Pub. No.: US 2008/0206828 A1
(43) Pub. Date: Aug. 28, 2008

(54) Device For Introducing Substance Into Cell, Cell Clamping Device and Flow Path Forming Method

(75) Inventors: Hidetoshi Kotera, Kyoto-shi (JP); Isaku Kanno, Kyoto-shi (JP); Takaaki Suzuki, Kyoto-shi (JP); Masao Washizu, Bunkyo-ku (JP)

Correspondence Address: SUGHRUE MION, PLLC, 2100 PENNSYLVANIA AVENUE, N.W., SUITE 800, WASHINGTON, DC, 20037, US

(73) Assignee: Kyoto University, Kyoto-shi, Kyoto-fu (JP)

(21) Appl. No.: 11/908,882

(22) PCT Filed: Mar. 17, 2006
(86) PCT No.: PCT/JP2006/305392

§ 371 Date: Dec. 12, 2007

(30) Foreign Application Priority Data
Mar. 17, 2005 (JP) 2005-077817

(51) Int. Cl. C12M 1/42; C12N 13/00; G03F 7/20

(52) U.S. Cl. 435/173.1; 435/285.2; 430/324

(57) ABSTRACT

A device for introducing a substance into a cell which can realize a high-efficient external substance introduction by means of electro-poration not depending on a cell size, a cell clamping device capable of clamping a cell at many locations, and a flow path forming method capable of efficiently forming a flow path. The device for introducing a substance into a cell (10a) comprises an insulating thin film (2) having a pore (1) and a pair of electrodes (6, 7) disposed on the opposite sides of the film (2) across the pore (1). When a cell (9) is fixed to the pore (1) and a pulse voltage is applied to between the electrodes (6, 7) with a space (5) filled with a fluid containing substance (4) to be introduced into the cell (9), a field concentration to a pore portion is used to destroy a cell membrane to thereby introduce the substance (4) into the cell (9).

### TECHNICAL FIELD

The present invention relates to a device for introducing a substance into a cell, a cell clamping device, and a flow path forming method, and more specifically, to a device for introducing a substance into a cell which introduces an external substance into a cell, a cell clamping device for evaluating functions of a cell, and a flow path forming method especially preferable for forming these devices.

### BACKGROUND ART

Conventionally, as a method for introducing an external substance such as genes into a cell, there is available a micro-injection method which uses glass capillary to inject an external substance into cells one by one, and an electro-poration method which reversibly destroys and permeabilizes a cell membrane by applying an electric field to a cell suspension to introduce an external substance into the cell.

According to the micro-injection method, an external substance can be reliably introduced into cells one by one, however, this requires complicated delicate control, so that it is difficult to handle a large amount of cells. On the other hand, the electro-poration method is suitable for handling a large amount of cells. For example, in Patent document 1, a chemical injector for a cell is disclosed.

As a method for measuring a cell's electrical properties, mechanical properties, and response to a mechanical stimulus, a patch clamp method is generally used. For example, a micro-pipet manufactured by hot-drawing-out a glass tube is brought into contact with a cell surface to measure changes in electrical properties of the cell surface.

Non-patent document 2 discloses a technique for forming a micro structure like a mesh, etc., by using a resist SU-8 (made by microchem) from which a structure with a high aspect ratio can be manufactured.
Patent document 1: Japanese Unexamined Patent Publication No. H10-337177
Non-patent document 1: U. Zimmermann, “Electrical breakdown, electropermeabilization and electrofusion,” Rev. Physiol. Biochem. Pharmacol. 105, p. 175-345 (1986)
Non-patent document 2: Hironobu Sato, Takayuki Kakinuma, Jeung Sang Go and Shuichi Shoji, “In-channel 3-D micromesh structures using maskless multi-angle exposures and their microfilter application,” Sensors and Actuators A: Physical, Volume 111, Issue 1, 1 Mar. 2004, Pages 87-92

### DISCLOSURE OF THE INVENTION

### Problem to be Solved by the Invention

The electro-poration method has the following problem.

A membrane voltage Vm(θ) to be applied to a cell membrane of a cell with a radius a placed in a uniform electric field E is obtained as:

Vm(θ)=1.5×a×E×cos θ (1)

Herein, θ indicates an angle between an electric flux line and a radius (refer to Non-patent document 1).

The equation (1) shows that the magnitude of the voltage to be applied to the cell membrane becomes maximum at the position of the most upstream side (north pole: θ=0) and the position of the most downstream side (south pole: θ=π) of the electric flux line, and its value is in proportion to the radius a of the cell. Therefore, when applying a uniform electric field pulse to the cell, the membrane is destroyed at the positions of the north pole and the south pole first.

It is known that when a pulse voltage appropriate for obtaining a membrane voltage of about 1V is applied, this destruction becomes a so-called reversible destruction, and the cell restores itself. In the process of this reversible destruction, the permeability of the membrane increases, so that an external substance around the cell is ingested into the inside of the cell membrane.

However, when an excessive voltage is applied, the destruction of the membrane is irreversible and the cell cannot restore itself, and the destruction propagates to the whole membrane, and as a result, the cell itself is destroyed and the cell dies.

This membrane voltage is in proportion to the radius a of the cell as shown in the equation (1). On the other hand, the diameters of the cells are not uniform, and normally have a distribution ranging to some extent. Therefore, when a uniform pulse electric field is applied to a suspension of many cells, a) on a large cell, an excessively great membrane voltage is generated and the cell itself is destroyed, and b) the membrane voltage to be applied to a small cell is excessively low and the membrane is not permeabilized, and an external substance is not introduced Specifically, the external substance is introduced into only cells in a certain range of size.

In view of these circumstances, an object of the present invention is to provide a device for introducing a substance into a cell which can realize high-efficient external substance introduction by means of electro-poration not depending on a cell size.

In the patch clamp method, the number of glass tubes that can be connected to a cell is 2 or 3 at most, and it is difficult to measure intracellular substance movement, intercellular substance movement, and action in a smaller cell and between smaller cells.

In view of these circumstances, an object of the present invention is to provide a cell clamping device which can clamp a cell at many portions.

Further, in the device for introducing a substance into a cell and the cell clamping device, it is necessary to form flow paths. Generally, a flow path is formed by joining a substrate having grooves in its main surface to another substrate. However, with this method, a joining defect is easily caused by warping of the substrates, and it is difficult to efficiently form a flow path.

In view of these circumstances, an object of the present invention is to provide a flow path forming method for efficiently forming a flow path.

### Means for Solving the Problems

In order to solve the above-described problems, the present invention provides a device for introducing a substance into a cell, constructed as follows.

The device for introducing a substance into a cell comprises an insulating thin film having a pore, and a pair of electrodes arranged on both sides of the insulating thin film so as to face the pore. The device for introducing a substance into a cell fixes a cell to the pore in one region with respect to the insulating thin film, and in a state that a space continuous to the pore in another region with respect to the insulating thin film is filled with a fluid including a substance desired to be introduced into the cell, the substance is introduced into the cell by destroying a cell membrane of the cell by applying a pulse voltage between the pair of electrodes.

In the construction described above, when a pulse voltage is applied between the pair of electrodes, an electric field concentrates at the pore provided in the insulating thin film, so that a cell membrane in contact with the pore is reversibly or irreversibly destroyed and the substance included in the fluid filling the space continuous to the pore can be introduced into the cell.

To obtain electric field concentration at the pore portion with respect to a voltage with a pulse width Specifically used in actuality for electro-poration, when the thickness of the insulating thin film is defined as d, the permittivity is defined as ε, the distance between the pair of electrodes is defined as L, and the resistivity of the fluid is defined as ρ, it is preferable that the thickness d and the permittivity ε of the insulating thin film, the distance L between the pair of electrodes, and the resistivity ρ of the fluid are selected so that a time constant τ=ερL/d becomes 10 milliseconds or less.

Preferably, the electrode placed on the side with the presence of the cell of the insulating thin film is placed at a position at a distance of 10 times or more the diameter of the pore from the pore. Thereby, an electric field can be sufficiently concentrated at the pore.

Preferably, the diameter of the pore is one-third or less of the diameter of the cell. In this case, an electric field can be sufficiently concentrated at a portion of the cell membrane in contact with the pore and only the portion of the cell membrane in contact with the pore can be permeabilized.

Preferably, the pulse voltage to be applied between the pair of electrodes is 10V or less. By using the pulse voltage of 10V or less, only the membrane of the pore portion can be permeabilized by using the electric field concentrating at the pore without damaging other portions of the cell membrane.

To fix the cell to the pore, suctioning fixation, sedimentation due to gravity, dielectrophoresis, or electrophoresis can also be used.

Preferably, in the other region with respect to the insulating thin film, the space continuous to the pore is hermetically sealed except for the pore portion. With the above-described construction, the space continuous to the pore is hermetically sealed except for the pore portion, so that by suctioning the fluid filled in the space, the fixation of the cell to the pore can be stabilized.

Preferably, surface modification to be bonded to the cell is applied around the pore of the insulating thin film. With the construction described above, propagation of destruction at the pore portion of the cell membrane to the surrounding can be actively prevented.

To solve the above-described problems, the present invention provides a cell clamping device constructed as follows.

The cell clamping device has a resin coating made of a photo-curing resin. This resin coating has a plurality of sets of an opening for fixing a cell, formed in one main surface of the resin coating, a pore continuous to the opening, and a communication hole communicating with the pore.

In the above-described construction, by suctioning a cell via the pores and communication holes, the cell can be fixed to the openings. A plurality of openings are provided, so that the cell can be clamped at many portions. The cell clamping device can be used for an operation for introducing an external substance into the cell and measurement of cell functions such as mechanical, electrical, chemical responses to a stimulus while clamping the cell.

Preferably, a substrate for supporting the resin coating is provided. On this substrate, a conductive film which extends along the communication hole from the bottom surface of the pore facing the opening is formed.

With the construction described above, the conductive film can be used as an electrode or electric wiring. For example, it can be used for applying a pulse voltage between the electrode arranged on the cell side and the conductive film and introducing a substance into the cell in a state that a fluid including the substance to be introduced into the cell is filled in the pore and the communication hole. The conductive film can also be used for measuring a potential and a current between the electrode disposed on the cell side and the conductive film.

Preferably, a columnar support member is further provided. The columnar support member includes a base arranged along the one main surface, projections projecting to the opposite sides of the openings from the base, and through holes that communicate the tip ends of the projections with the openings.

In the construction described above, the cell can be supported by the tip ends of the projections of the columnar support member while floating from the base of the columnar support member. In this case, the cell can be suctioned and fixed via the through holes and the pores of the resin coating and the communication holes of the columnar support member.

With the construction described above, in addition to a response when instantaneously introducing the external substance into the cell from the tip end of the projection of the columnar support member (intracellular concentration jump), a response when instantaneously introducing a reagent with a constant concentration or molecules into the surrounding of the cell (extracellular concentration jump) can be measured. As a response, in addition to a potential and a current value of each projection of the columnar support member, a warp (deformation and displacement) of the projection accompanying the response can be measured.

Further, in order to solve the above-described problems, the present invention provides a flow path forming method constructed as follows.

The flow path forming method comprises first through fourth steps. At the first step, a light shielding pattern which blocks light transmission is formed on at least one main surface of a transparent substrate. At the second step, a photo-curing resin is coated onto the one main surface or the other main surface of the substrate. At the third step, from the opposite side of the photo-curing resin with respect to the substrate, the substrate is irradiated with light at different angles, and in the photo-curing resin, the light is transmitted through a region other than a non-transmitting region extending along the light shielding pattern to cure the portion of the photo-curing resin through which the light was transmitted. At the fourth step, the non-transmitting region of the photo-curing resin is removed. At the first step, the light shielding pattern includes at least one of a narrow and long first portion and a second portion Specifically continuous to the first portion and extends in a direction substantially perpendicular to the extending direction of the first portion. At the third step, the non-transmitting region includes at least one of a first region corresponding to the first portion of the light shielding pattern and a second region corresponding to the second portion of the light shielding pattern. The first region extends to the substrate side in the photo-curing resin. The second region extends from the substrate side in the photo-curing resin to the main surface of the photo-curing resin on the opposite side of the substrate. At the fourth step, a horizontal hole extending along the substrate is formed by removing the first region of the non-transmitting region, and a vertical hole having an opening in the main surface of the photo-curing resin on the opposite side of the substrate is formed by removing the second region of the non-transmitting region.

In the method described above, the light shielding pattern may have only the first portion or second portion, a plurality of first portions, or a plurality of second portions. Further, it may have a plurality of pairs of first and second portions. A flow path formed of either one of the horizontal hole or the vertical hole may be formed. In the method described above, a flow path can be formed more easily than in the case where a substrate with grooves formed in its main surface is joined to another substrate.

Preferably, at the first step, on one main surface of the substrate, the light shielding pattern is formed by using a conductive material. At the second step, the photo-curing resin is coated onto the one main surface of the substrate. In this case, a conductive pattern extending along the flow path from a portion facing the openings of the pores can be formed at the same time. This conductive pattern can be used as an electrode or electric wiring.

Further, in order to solve the above-described problems, the present invention provides a flow path forming method constructed as follows.

The flow path forming method comprises first through fourth steps. At the first step, a photo-curing resin is coated onto one main surface of a transparent substrate. At the second step, along the other main surface of the substrate, a mask member having a light shielding pattern is arranged along the other main surface of the substrate. At the third step, from the mask member side, the mask member is irradiated with light at different angles, and in the photo-curing resin, the light was transmitted through a region other than a non-transmitting region extending along the light shielding pattern to cure the portion of the photo-curing resin through which the light is transmitted. At the fourth step, the non-transmitting region of the photo-curing resin is removed. At the first step, the light shielding pattern includes at least one of a narrow and long first portion and a second portion Specifically continuous to the first portion and extends in a direction substantially perpendicular to the extending direction of the first portion. At the third step, the non-transmitting region includes at least one of a first region corresponding to the first portion of the light shielding pattern and a second region corresponding to the second portion of the light shielding pattern. The first region extends to the substrate side in the photo-curing resin. The second region extends from the substrate side in the photo-curing resin to a main surface of the photo-curing resin on the opposite side of the substrate. At the fourth step, a horizontal hole extending along the substrate is formed by removing the first region of the non-transmitting region, and a vertical hole having an opening in the main surface of the photo-curing resin on the opposite side of the substrate is formed by removing the second region of the non-transmitting region.

### Effects Of the Invention

According to the device for introducing a substance into a cell of the present invention, electro-poration can be performed regardless of a size of the cell, so that high-efficient external substance introduction is realized. In addition, by arranging many pores on the insulating thin film, an external substance can be concurrently introduced into a large amount of cells.

According to the cell clamping device of the present invention, a cell can be clamped at many portions.

Further, according to the flow path forming method of the invention, a flow path can be efficiently formed.

### BRIEF DESCRIPTION OF THE DRAWINGS

FIG. 1 is a sectional view showing a construction of a device for introducing a substance into a cell (Example 1);

FIG. 2 is a sectional view showing a construction of a device for introducing a substance into a cell (Example 2);

FIG. 3 is an explanatory view showing a minimum constituent unit of the cell clamping device (Example 3);

FIG. 4 is an explanatory view of irradiation (Example 3);

FIG. 5 is a sectional view showing a construction of a cell clamping device (Example 4);

FIG. 6 is a plan view showing a construction of a cell clamping device (Example 5);

FIG. 7 is a perspective view showing a construction of a columnar support member (Example 6);

FIG. 8 is a main portion sectional view showing a construction when a columnar support member is used for the cell clamping device (Example 6);

FIG. 9 is an explanatory view of measurement using the columnar support member for the cell clamping device (Example 6); and

FIG. 10 is an explanatory view of a method for manufacturing the columnar support member (Example 6).

### DESCRIPTION OF THE REFERENCE NUMERALS

1 pore
2 insulating thin film
6, 7 electrode
9 cell
10a, 10b device for introducing substance into cell
11 insulating thin film
12 pore
14, 15 electrode
20 cell clamping device
21 substrate
22, 22a, 22b conductive film
23 resin coating
24s opening
24a, 24b, 24t pore (vertical hole)
25a, 25b, 25t communication hole (horizontal hole)
30 cell clamping device
32 substrate
33 resin coating
34a-34h pore (vertical hole)
35a-35h communication hole (horizontal hole)
50 columnar support member
52 base
54 projection
54a tip end
56 through hole
80 cell

### BEST MODE FOR CARRYING OUT THE INVENTION

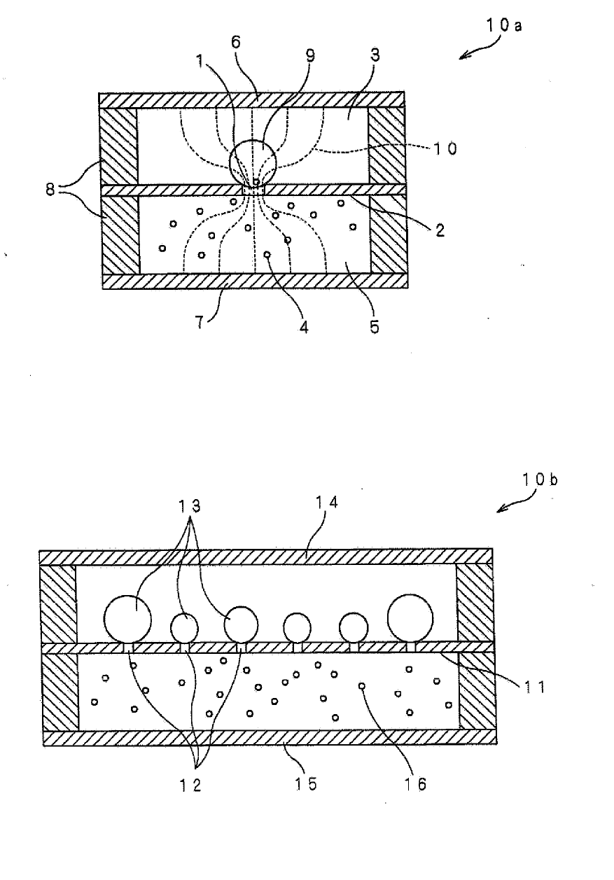

Hereinafter, examples as embodiments of the present invention will be described with reference to FIG. 1 through FIG. 10.

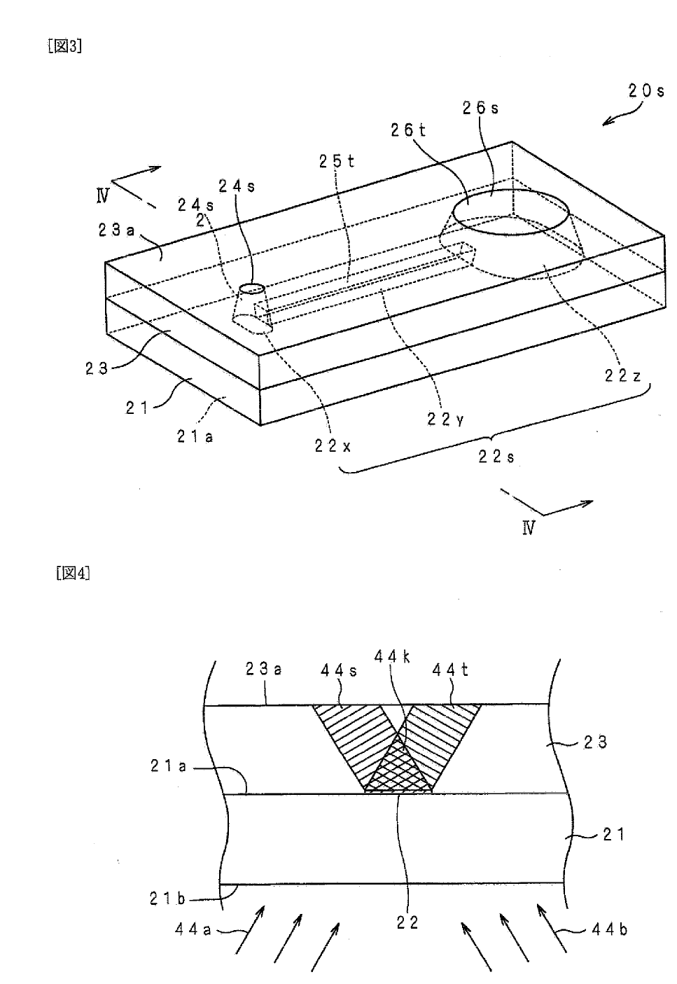

First, a device for introducing a substance into a cell will be described with reference to FIG. 1 and FIG. 2.

As shown in FIG. 1, in a device 10a for introducing a substance into a cell, on both sides of an insulating thin film 2 having a pore 1, a chamber 3 filled with a buffer (for example, normal saline solution) and a chamber 5 filled with a solution of an external substance 4 are provided, and are in contact with electrodes 6 and 7, respectively. The reference numeral 8 denotes a spacer for holding the electrode, and commonly serves as a chamber wall in conjunction with the electrode. To the side of the chamber 3 filled with a buffer of the pore 1, a cell 9 is suctioned and fixed, and a pulse voltage is applied between the electrodes 6 and 7. The insulating thin film 2 must separate the fluid between the electrodes 6 and 7, and in particular, when the chamber 5 is hermetically sealed except for the portion of the pore 1, it becomes easy to suction and fix the cell 9 to the pore 1, and liquid leakage from the chamber 5 is prevented.

The insulating thin film 2 is, for example, an SiO₂ film. In detail, by thermally oxidizing an Si substrate, an SiO₂ film is formed, and the SiO₂ film is coated with a photoresist and a mask pattern is transferred thereon, and then development is performed, the portion that becomes the pore 1 of the SiO₂ film is etched, and finally, the Si substrate is removed by etching or the like, whereby an insulating thin film 2 of the SiO₂ film is obtained. It is also allowed that the Si substrate is not entirely removed but is partially left, and the insulating thin film 2 is supported by the remaining Si substrate.

Alternatively, an organic thin film having a pore opened by means of a laser or photolithography may also be used as the insulating thin film 2.

In FIG. 1, as shown by the dotted line, when the thickness of the insulating thin film 2 is defined as d, the permittivity is defined as ε, the distance between the pair of electrodes 6 and 7 is defined as L, and the electric resistance of the fluid in the chambers 3 and 5 is defined as ρ, under conditions that the thickness d of the insulating thin film, the permittivity ε, the distance L between the pair of electrodes 6 and 7, and the electric resistance ρ of the fluid are selected so that a time constant τ=ερL/d of the system becomes 10 milliseconds or less, the electric flux line 10 cannot pass through the insulating thin film 2, so that it passes through the pore 1. Specifically, the electric flux line 10 is converged, so that the electric field strength at the position of the pore becomes remarkably greater than in the solution. Therefore, only at the portion of the cell membrane in contact with the pore, electro-poration, Specifically, membrane permeabilization occurs, and the external substance 4 in the chamber 5 is introduced into the cell due to diffusion or electrophoresis.

To permeabilize only the portion of the cell membrane in contact with the pore by obtaining sufficient electric field concentration, it is desirable that the pore is sufficiently smaller than the diameter of the cell, more specifically, one-third or less of the diameter of the cell. In this case, the membrane voltage to be applied to the cell membrane at the portion in contact with the pore is determined by only the electric field concentration at the pore portion, and does not depend on the diameter of the cell. Specifically, by using this device, introduction of an external substance without depending on the diameter of the cell becomes possible.

Herein, by selecting an appropriate pulse voltage of 10V or less between the pair of electrodes 6 and 7, the membrane of the portion in contact with the pore 1 is reversibly destroyed and then restores itself, so that an external substance can be introduced into the cell without imparting great damage on the cell. In the case of normal electro-poration in which a pulse voltage is applied to a cell suspended in a solution, partial destruction of the cell membrane propagates to the entire cell and leads to destruction and death of the entire cell, however, in the case of this device, the cell membrane is fixed to the insulating thin film, so that the destruction occurring from the pore portion hardly propagates. To further actively prevent this propagation, application of a cell-adhering surface modification to the thin film around the pore is effective, and as a substance for this surface modification, for example, a cell-adhering modifier BAM (Biocompatible Anchor for Membrane: NOF Corporation), poly-D-lysine, phosphates, and FBS (fetal bovine serum) are exemplified. From this fact, according to this method, a permissible range of the voltage applicable without causing death of the cell is wide, and as a result, highly efficient introduction of an external substance can be performed.

The device for introducing a substance into a cell of the present invention is not limited to the structure shown in FIG. 1, and it is allowed that the chamber wall is not formed by the spacer 8 and the electrodes 6 and 7 as long as a solution which the two electrodes are in contact with or the electrodes are immersed in is present on both sides of the insulating thin film having a pore, and even in a structure having a chamber 3 and a chamber 5 that are not electrically insulated, electric flux line concentration occurs, so that the present invention is also applicable to this structure. Without limiting to suctioning, sedimentation by means of gravity, electrophoresis, or dielectrophoresis can also be used for fixing the cell.

FIG. 2 shows an example of concurrent parallel introduction of an external substance into cells. Many pores 12 are made in the insulating thin film 11, cells 13 are fixed to the pores, and when a pulse voltage is applied between the electrodes 14 and 15, the external substance 16 can be concurrently introduced into the cells. Therefore, large-amount parallel measurement and simultaneous measurement of cell membrane potentials are possible.

According to the device for introducing a substance into a cell of the present invention, electro-poration that does not depend on the diameter of a cell is realized, so that even when cells have a wide particle size distribution, highly efficient introduction of an external substance can be performed. The solution composition inside and outside the cell is arbitrary, so that substances except for substances which have a great conductance and do not have membrane permeability and caged substances (developed due to excitation and separation caused by light, etc.) can be measured.

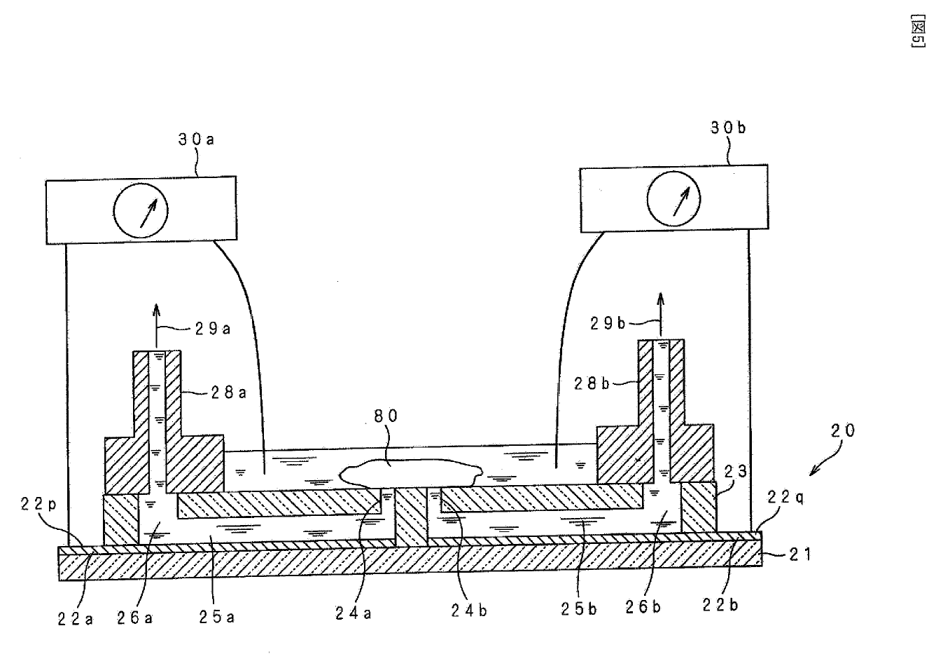

Next, a cell clamping device will be described with reference to FIG. 3 through FIG. 10.

FIG. 3 shows a main construction of a minimum constituent unit 20s of the cell clamping device.

The cell clamping device has a resin coating 23 made of an insulating resin arranged on an upper surface 21a of a transparent substrate 21.

On the upper surface 21a of the substrate 21, a conductive film 22 which blocks light transmission and has conducting properties is formed. The conductive film 22 includes a narrow and long first portion 22y and substantially oval second portions 22x and 22z which are continuous to both ends of the first portion 22y and extend in a direction substantially perpendicular to the extending direction of the first portion 22y. The conductive film 22 may include other portions connected to the first portion 22y or the second portions 22x and 22z.

In the upper surface 23a of the resin coating 23, openings 24s and 26s are formed, and for example, a cell is fixed to one opening 24s and suctioned from the other opening 26s by a syringe pump or a vacuum pump. In the resin coating 23, pores 24t and 26t continuous to the openings 24s and 26s and a communication hole 25t which communicate with the pores 24t and 26t are formed. The pores 24t and 26t are formed corresponding to the respective second portions 22x and 22z of the conductive film 22, and the second portions 22x and 22z of the conductive film 22 become bottom surfaces of the pores 24t and 26t. The communication hole 25t is formed corresponding to the first portion 22y of the conductive film 22, and the first portion 22y of the conductive film 22 becomes a bottom surface of the communication hole 25t.

Next, a production method will be described with reference to FIG. 4. FIG. 4, briefly, is a sectional view in the extending direction of the narrow and long first portion 22y of the conductive film 22.

First, on the upper surface 21a of a transparent substrate 21, a conductive film 22 having a predetermined pattern is formed. In detail, a glass substrate is used as the substrate 21. As the substrate 21, an Si substrate or a resin substrate may be used instead. The conductive film 21 is formed by coating the substrate 21 with a photoresist, transferring a mask pattern thereon and then developing it, and depositing Al thereon. A metal other than Al, such as Pt, Au, or Ag may be deposited. A method other than deposition may also be used.

Next, the upper surface 21a of the substrate 21 is coated with a photo-curing resin coating 23 (in detail, a negative resist SU-8 made by microchem).

Next, as shown by the arrows 44a and 44b, from the lower surface 21b side of the substrate 21 (Specifically, the opposite side of the resin film 23 with respect to the substrate 21), by irradiating the lower surface 21b of the substrate 21 with light at different angles, a portion which the light penetrated through of the resin coating 23 is cured.

At this time, the light irradiating directions 44a and 44b are two directions which are inclined from the lower surface 21b of the substrate 21 and are substantially symmetric to each other when viewed in the extending direction of the narrow and long first portion 22y of the conductive film 22. When light is irradiated in the direction shown by the arrow 44a, inside the resin coating 23, a region 44t which falls under shadow of the conductive film 22 and the light does not penetrate through is formed. On the other hand, when light is irradiated in the direction shown by the arrow 44b, inside the resin coating 23, a region 44s which falls under shadow of the conductive film 21s and the light does not penetrate through is formed. A region 44k in which the two shadow regions 44s and 44t overlap each other is a non-transmitting region which light does not penetrate through at all. In the resin coating 23, a portion other than the non-transmitting region 44k is cured.

In detail, in a state that the substrate 21 is inclined by ±45 degrees from an ultraviolet ray irradiation direction, ultraviolet parallel light is irradiated onto the resin coating 23 from the substrate 21 side by using an exposure apparatus, and then the resin coating is heated to a temperature (for example, 95 degrees C.) higher than a temperature at which the SU-8 of the resin coating 23 starts curing.

Next, the non-transmitting region 44k of the resin coating 23 is removed. When the resin coating 23 is SU-8, it is soaked in a developer to elute the non-transmitting region 44k of the resin coating 23.

The non-transmitting region 44k extends along the conductive film 22. The non-transmitting region 44k includes a first region corresponding to the first portion 22y of the conductive film 22 and second regions corresponding to the second portions 22x and 22z of the conductive film 22.

The first portion 22y of the conductive film 22 is narrow in width, so that the first region of the non-transmitting region is in a triangular sectional shape inside the resin coating 23 and extends to only the glass substrate 21 side in the resin coating 23. Therefore, corresponding to the first region of the non-transmitting region 44k, a communication hole (horizontal hole) 25t shaped like a tunnel having a triangular sectional shape is formed.

On the other hand, the widths of the second portions 22x and 22z of the conductive film 22 are wide, so that the second regions of the non-transmitting region 44k become trapezoid in a section reaching the upper surface 23a of the resin coating 23 inside the resin coating 23, and extend from the substrate 21 side to the upper surface 23a on the opposite side inside the resin coating 23. Therefore, openings 24s and 26s are formed in the upper surface 23a of the resin coating 23, and corresponding to the second regions of the non-transmitting region 44k, pores (vertical holes) 24t and 26t in roughly circular truncated cone shape are formed.

For example, a glass substrate is used as the substrate 21, the SU-8 is used as the resin coating 23, and Al is deposited as the conductive film 22, whereby a cell clamping device having the resin coating 23 with a coating thickness of 10 micrometers, the communication hole 25t with a width of 5 micrometers, the opening 24s with a diameter of 3 micrometers, and the opening 26s with a diameter of 6 micrometers can be manufactured.

In the above-described production method, in comparison with a general micro-channel (flow path) forming method involving chemical and physical etching of a substrate and joining of substrates having grooves, large-scale production equipment is not necessary, the production process is easier, and the production cost is less. Therefore, a flow path can be efficiently formed. Along with formation of the flow path, electrodes and electric wiring can also be formed.

The cell clamping device has a plurality of pores and communication holes, so that it can be used for various purposes while a plurality of portions of a cell are fixed to the pores.

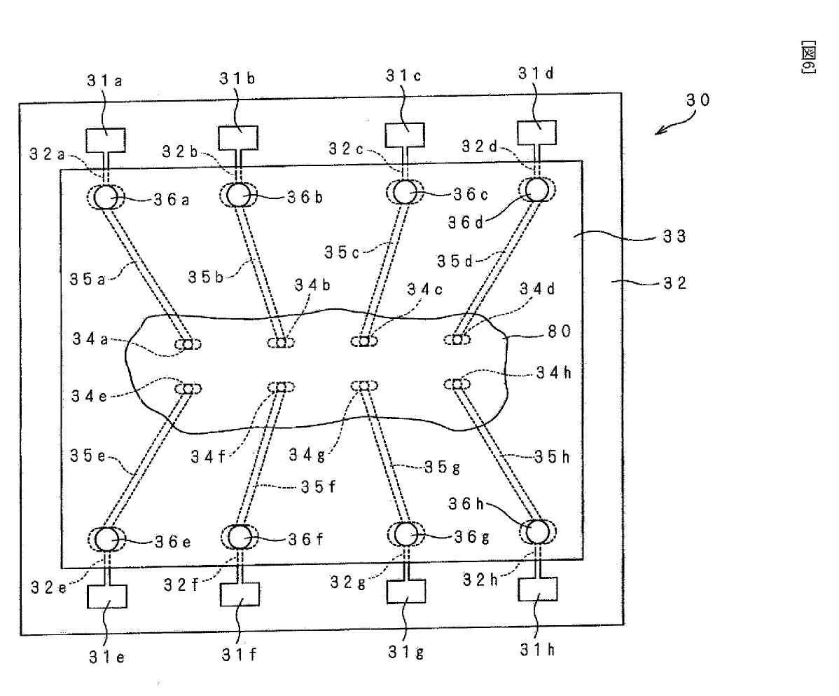

As shown in FIG. 5, when the cell clamping device 20 is provided with a first set of pores 24a and-26a and a communication hole 25a and a second set of pores 24b and 26b and a communication hole 25b, two portions of a cell 80 are fixed to the pores 24a and 24b by suctioning with syringe pumps 28a and 28b provided in the pores 26a and 26b as shown by the arrows 29a and 29b, and the currents flowing through the pores 24a and 24b are measured with ammeters 30a and 30b via the conductive films 22a and 22b and the external terminals 22p and 22q.

The cell clamping device 20 can be used as a device for introducing a substance into a cell by arranging an electrode above the cell. For example, an external substance is introduced from one portion of the cell and a response at another portion of the cell is detected, whereby gap-junctional substance transportation (passing of ions and small molecules (cAMP, cGMP, etc.)) can be measured.

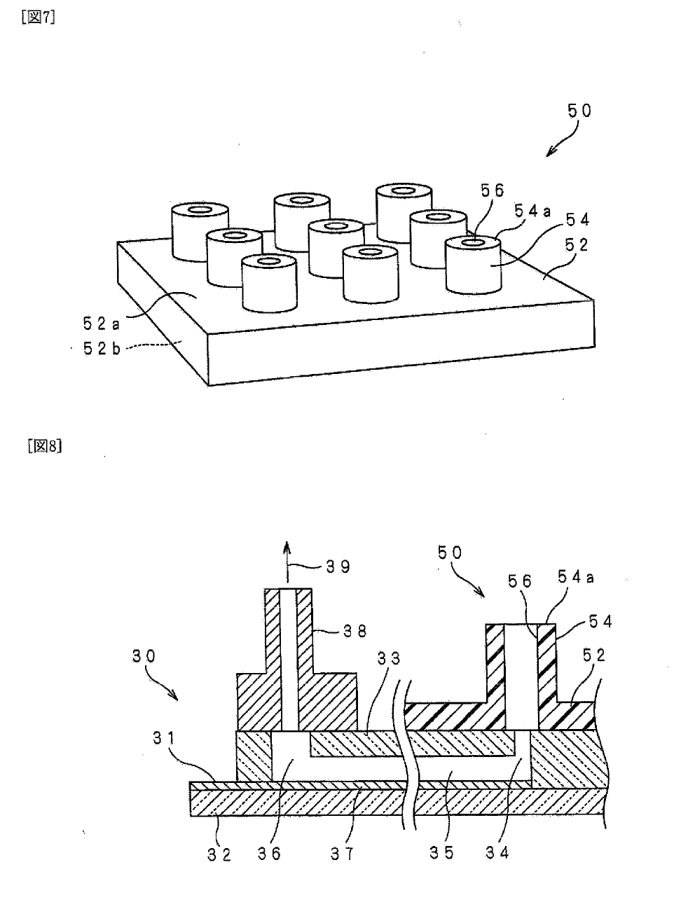

FIG. 6 shows a construction of a cell clamping device 30 provided with many pores 34a through 34h.

In the cell clamping device 30, similar to the cell clamping device 20s of the minimum constituent unit, on a transparent glass substrate 32, a resin coating 33 made of an insulating resin smaller than the glass substrate 32 is disposed. In the resin 33, a plurality of sets of cell fixing pores 34a through 34h, communication holes 35a through 35h, and suctioning pores 36a through 36h are formed. A conductive film to be formed on the glass substrate 32 includes portions that form the cell fixing pores 34a through 34h, communication holes 35a through 35h, and suctioning pores 36a through 36h, and in addition, extended portions 32a through 32h extended from said portions and external terminals 31a through 31h provided at the ends of the extended portions 32a through 32h.

In the cell clamping device 30, by irradiating the resin coating 33 made of a photo-curing resin with light at different angles, the cell fixing pores 34a through 34h, communication holes 35a through 35h, and suctioning pores 36a through 36h are formed, and at the same time, corresponding to the extended portions 32a through 32h of the conductive film formed on the glass substrate 32, flow paths are formed. Therefore, it is preferable that the extended portions 32a through 32h are as narrow as possible in width. The flow paths that are formed corresponding to the extended portions 32a through 32h are preferably closed by pressing the resin coating 33 with clips or the like.

The cell clamping device 30 can be used for various measurements and operations of the cell 80 while clamping a plurality of portions of the cell 80.

The cell clamping device can be used together with a columnar support member 50 as shown in FIG. 7 through FIG. 10.

As shown in FIG. 7, the columnar support member 50 includes a plate-shaped base 52, a plurality of columnar projections 54 projecting from the upper surface 52a of the base 52, and through holes 56 perforating from the tip ends 54a of the respective projections 54 to the lower surface 52b of the base 52.

The columnar support member 50 can be manufactured according to, for example, the method shown in FIG. 10. Specifically, as shown in FIG. 10(a), a ring-shaped light shielding pattern 62 is formed on one main surface 60a of a transparent glass substrate 60. Then, as shown in FIG. 10(b), the upper surface 60a of the glass substrate 60 is spin-coated with a photo-curing resin 64 (for example, negative resist SU-8), and as shown by the arrow 61, the lower surface 60b of the glass substrate 60 is exposed to parallel light perpendicularly from the lower surface 60b side of the glass substrate 60 to cure the portions which the light penetrated through inside the resin 64. Then, as shown in FIG. 10(c), the unexposed portions of the resin 64 are removed to form cylindrical spaces 65. Next, as shown in FIG. 10(c), the upper surface 64a of the resin 64 is coated with a resin 66 (for example PDMS: polydimethylsiloxane) to a predetermined thickness, and after filling the resin 66 into the cylindrical spaces 65, the resin 66 is cured. Then, as shown in FIG. 10(d), the resin 66 is separated from the resin 64. At this time, in the resin 66, cylindrical projections 67 are formed, however, the central holes 68 of the projections 67 are not perforated, so that last, the bottoms of the central holes 68 are machined with a laser to perforate the central holes 68.

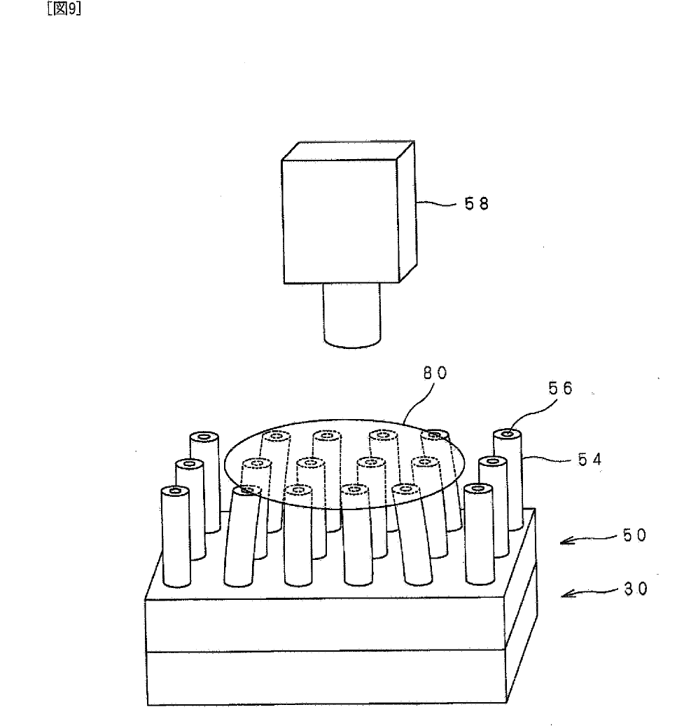

As shown in FIG. 8, the columnar support member 50 is arranged along the resin coating 33 of the cell clamping device 30. At this time, each projection 54 of the columnar support member 50 faces the opening of the pore 34 formed in the upper surface of the resin coating 33 via the base 52, and the through hole 56 of the columnar support member 50 communicates with the pore 34 of the cell clamping device 30. By suctioning as shown by the arrow 39 by the syringe pump 38 from the pore 36 of the cell clamping device 30, a cell can be adsorbed to the tip end 54a of the projection 54 of the columnar support member 50 via the communication hole 35, the pore 34, and the through hole 56.

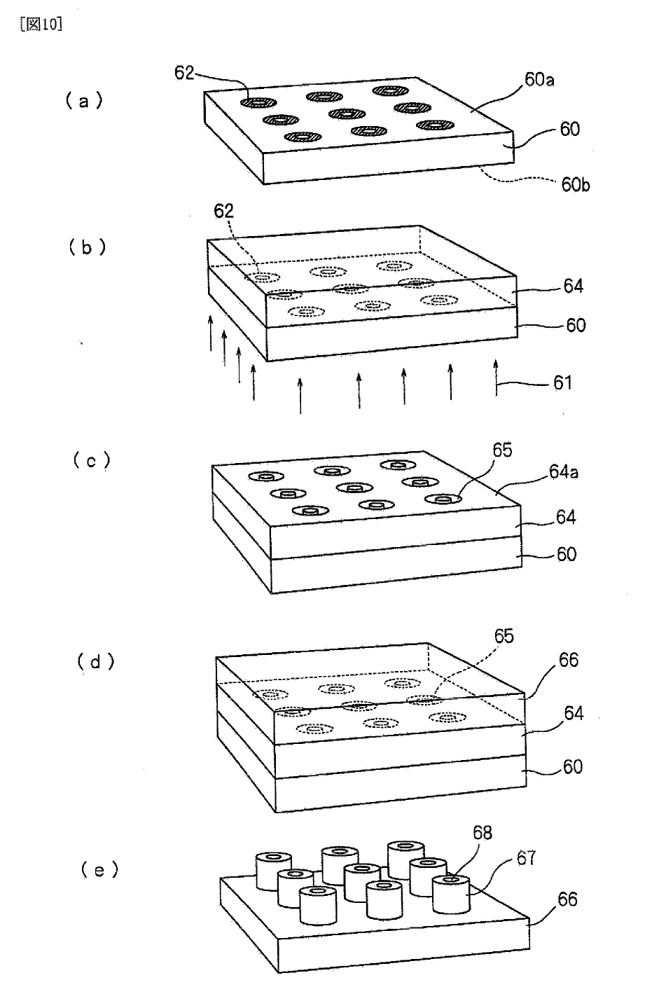

At this time, as shown in FIG. 9, while floating from the base 52 of the columnar support member 50, the cell 80 is supported at a plurality of portions by projections 54 of the columnar support member 50. The projections 54 of the columnar support member 50 are constructed so as to have appropriate rigidity and images of displacements of the tip ends of the projections 54 are taken with a camera 58 and the taken images are analyzed, whereby the displacements of the respective portions of the cell 80 are measured. Alternatively, currents flowing through the supported portions of the cell 80 are measured via the conductive film 37 and the external terminal 31. For example, a reagent with a constant concentration or molecules are instantaneously introduced into the surrounding of the cell 80 and a response (extracellular concentration jump) of the cell 80 at this time is measured.

By combining the columnar support member 50 and the cell clamping device 30, a plurality of portions of the cell 80 can be stably supported. By adsorbing the cell to the tip ends 54 of the projections 54 of the columnar support member 50, the supported portions of the cell 80 can be supported so as not to be displaced. Due to warping of the projections 54 of the columnar support member 50, restrictions on the cell 80 is less, and the cell 80 can move with a high degree of freedom. Therefore, the states of the portions of the cell 80 can be accurately measured.

As described above, the device for introducing a substance into a cell can perform electro-poration regardless of the size of the cell, so that it realizes high-efficiency introduction of an external substance. By arranging a number of pores on an insulating thin film, concurrent parallel introduction of an external substance into a large amount of cells is realized.

The cell clamping device can clamp a cell at many portions.

Further, according to a flow path forming method for manufacturing the cell clamping device, flow paths can be efficiently formed. Without limiting to the cell clamping device, this flow path forming method can be applied to various fields.

The present invention is not limited to the above-described embodiments, but can be carried out while variously revised.

For example, instead of the negative resist, a positive resist can be used as the photo-curing resin. In this case, the light shielding pattern is reversed. The light shielding pattern may be other than the conductive film, and may be removed after forming flow paths.

It is also allowed that the light shielding pattern for forming flow paths is formed on the other main surface of a transparent substrate whose one main surface is coated with a photo-curing resin, and the photo-curing resin is irradiated with light at different angles via the light shielding pattern. It is also allowed that a mask member having a light shielding pattern is arranged along the other main surface of a transparent substrate whose one main surface is coated with a photo-curing resin, and the photo-curing resin is irradiated with light at different angles via the light shielding pattern of the mask member. In both of these cases, the non-transmitting region which the irradiated light does not penetrate through extends along the light shielding pattern while separated from the light shielding pattern.

### INDUSTRIAL APPLICABILITY

The device for introducing a substance into a cell and the cell clamping device are devices which renew the research methods in the fields of molecular biology and science, frontier medical science, medical treatment, and drug discovery, and at the same time, enable point-of-care and tailor-made medical treatment easily and less-invasively. The flow path forming method of the present invention can be used for manufacturing microdevices and micro structures using MEMS technologies such as μTAS (Total Analysis Systems).

1-13. (canceled)

14. A method of introducing a substance into a cell, comprising:
a first step of arranging a pair of electrodes on both sides of an insulating thin film having a pore, fixing a cell to the pore in one region with respect to the insulating thin film, and filling a space continuous to the pore in another region with respect to the insulating thin film with a fluid including a substance to be introduced into the cell;
a second step of destroying only a portion contacting the pore among a cell membrane of the cell fixed to the pore so as to introduce the substance into the cell from the space continuous to the pore by using electric field concentration to a position of the pore caused by applying a pulse voltage of less than 10 volts between the pair of electrodes.

15. A device for introducing a substance into a cell to be used by the method of claim 14, comprising:
an insulating thin film having a pore;
a first chamber to be filled with a buffer including a cell, continuing from the pore in one region with respect to the insulating thin film;
a second chamber to be filled with a fluid including a substance to be introduced into the cell, continuing from the pore in anther region with respect to the insulating thin film; and
a pair of electrodes arranged on both sides of the insulating thin film so that one of the pair of electrodes contacts the buffer filled in the first chamber and another of the pair of electrodes contacts the fluid filled in the second chamber, wherein
the insulating thin film is constructed so that: in a state that the cell in the first chamber is fixed to the pore in one region with respect to the insulating thin film and the second chamber continuous to the pore in another region with respect to the insulating thin film is filled with the fluid including the substance to be introduced into the cell, the electric field strength at a position of the pore is caused to be remarkably greater than the electric field strength in the buffer and the electric field strength in the fluid and thereby electric field concentration is caused to be strong enough to destroy only a portion contacting the pore among a cell membrane of the cell fixed to the pore so as to introduce the substance into the cell from the second chamber continuous to the pore by applying a pulse voltage of less than 10 volts between the pair of electrodes.

16. The device for introducing a substance into a cell according to claim 15, wherein when the thickness of the insulating thin film is defined as d, the permittivity is defined as ε, the distance between the pair of electrodes is defined as L, and the electric resistance of the fluid is defined as ρ, the thickness d and the permittivity ε of the insulating thin film, the distance L between the pair of electrodes, and the electric resistance ρ of the fluid are selected so that a time constant of the system τ=ερL/d becomes 10 milliseconds or less.

17. The device for introducing a substance into a cell according to claim 15, wherein the one of the pair of electrodes, arranged on the side with the presence of the cell of the insulating thin film, is placed at a distance of 10 times or more diameter of the pore from the pore.

18. The device for introducing a substance into a cell according to claim 15, wherein the diameter of the pore is one-third or less of the diameter of the cell to be fixed to the pore.

19. The device for introducing a substance into a cell according to claims 15, wherein in the other region with respect to the insulating thin film, the second chamber continuous to the pore is hermetically sealed except for a border with the pore.

20. The device for introducing a substance into a cell according to claims 15, wherein surface modification to be bonded to the cell is applied to the surrounding of the pore of the insulating thin film.

21. A cell clamping device comprising:
a resin coating made of a photo-curing resin; and
a substrate which supports the resin coating; wherein
the resin coating comprises a plurality of sets of:
an opening for fixing a cell, formed in one main surface of the resin coating;
a pore continuous to the opening, reaching down to another main surface of the resin coating; and
a communication hole which communicates with the pore, bounding on the other main surface of the resin coating, and wherein
the substrate is provided with a conductive film on a surface thereof supporting the resin coating so that the conductive film forms a bottom surface of the pore and a bottom surface of the communication hole.

22. A cell clamping device comprising:
(1) a resin coating made of a photo-curing resin, comprising a plurality of sets of:
an opening for fixing a cell, formed in one main surface of the resin coating;
a pore continuous to the opening; and
a communication hole which communicates with the pore;
(2) a substrate which supports the resin coating, provided with a conductive film so that the conductive film extends along a bottom surface of the communication hole from a bottom surface of the pore facing the opening; and
(3) a columnar support member which includes:
a base arranged along the one main surface of the resin coating;
projections which face the openings of the resin coating via the base and project to the opposite side of the openings of the resin coating from the base; and
through holes that communicate the tip ends of the projections and the openings with each other, wherein
in a state that the columnar support member supports a cell by adsorbing plural portions of the cell to the tip ends of the projections of the columnar support member respectively, the cell is able to move due to deflection of the projections of the columnar support member.

23. A flow path forming method comprising:
a first step of forming a light shielding pattern which blocks light transmission on at least one main surface of a transparent substrate;
a second step of coating at least one main surface of the substrate with a photo-curing resin;
a third step of irradiating the substrate with the light at different angles from the opposite side of the photo-curing resin with respect to the substrate so as to transmit the light through a region other than a non-transmitting region extending along the light shielding pattern inside the photo-curing resin, and thereby curing the region of the photo-curing resin through which the light is transmitted; and
a fourth step of removing the non-transmitting region of the photo-curing resin, wherein
at the first step, the light shielding pattern includes: a first portion having relatively long length in an extending direction of the first portion and relatively short width in a direction substantially perpendicular to the extending direction of the first portion; and a second portion which is continuous to the first portion and extends in a direction substantially perpendicular to the extending direction of the first portion,
at the third step:
the non-transmitting region includes a first region corresponding to the first portion of the light shielding pattern and a second region corresponding to the second portion of the light shielding pattern;
only one of the first region of the non-transmitting region is formed corresponding to one of the first portion of the light shielding pattern;
the first region extends to the substrate side inside the photo-curing resin, and a cross section of the first region substantially perpendicular to the extending direction of the first region has a triangle shape, one side of which bounds on the light shielding pattern; and
the second region extends from the substrate side inside the photo-curing resin to a main surface opposite to the substrate of the photo-curing resin, and
at the fourth step:
a horizontal hole is formed like a tunnel having a triangular sectional shape, one side of which bounds on the light shielding pattern, by removing the first region of the non-transmitting region; and
a vertical hole having an opening in the main surface opposite to the substrate of the photo-curing resin is formed by removing the second region of the non-transmitting region.

24. The flow path forming method according to claim 23, wherein
at the first step, the light shielding pattern is formed on the one main surface of the substrate by using a conductive material, and
at the second step, the one main surface of the substrate is coated with the photo-curing resin.

25. A flow path forming method comprising:
a first step of coating one main surface of a transparent substrate with a photo-curing resin;
a second step of arranging a mask member having a light shielding pattern along another main surface of the substrate;
a third step of irradiating the mask member with the light at different angles from the mask member side so as to transmit the light through a region other than a non-transmitting region extending along the light shielding pattern inside the photo-curing resin, and thereby curing the region of the photo-curing resin through which the light is transmitted; and
a fourth step of removing the non-transmitting region of the photo-curing resin, wherein
at the first step, the light shielding pattern includes a first portion having relatively long length in an extending direction of the first portion and relatively short width in a direction substantially perpendicular to the extending direction of the first portion and a second portion which is continuous to the first portion and extends in a direction substantially perpendicular to the extending direction of the first portion,
at the third step:
the non-transmitting region includes a first region corresponding to the first portion of the light shielding pattern and a second region corresponding to the second portion of the light shielding pattern;
corresponding to one of the first portion of the light shielding pattern, only one of the first region of the non-transmitting region is formed;
the first region extends to the substrate side inside the photo-curing resin, and a cross section of the first region substantially perpendicular to the extending direction of the first region has a triangle shape; and
the second region extends from the substrate side inside the photo-curing resin to a main surface opposite to the substrate of the photo-curing resin, and
at the fourth step:
a horizontal hole extending along the substrate is formed like a tunnel having a triangular sectional shape by removing the first region of the non-transmitting region; and
a vertical hole having an opening in the main surface opposite to the substrate of the photo-curing resin is formed by removing the second region of the non-transmitting region.

26. A flow path forming method comprising:
a first step of forming a light shielding pattern which blocks light transmission on at least one main surface of a transparent substrate;
a second step of coating at least one main surface of the substrate with a photo-curing resin;
a third step of irradiating the substrate with the light at different angles from the opposite side of the photo-curing resin with respect to the substrate so as to transmit the light through a region other than a non-transmitting region extending along the light shielding pattern inside the photo-curing resin, and thereby curing the region of the photo-curing through which the light is transmitted; and
a fourth step of removing the non-transmitting region of the photo-curing resin, wherein
at the first step, the light shielding pattern includes a first portion having relatively long length in an extending direction of the first portion and relatively short width in a direction substantially perpendicular to the extending direction of the first portion,
at the third step:
the non-transmitting region includes a first region corresponding to the first portion of the light shielding pattern;
only one of the first region of the non-transmitting region is formed corresponding to one of the first portion of the light shielding pattern;
the first region extends to the substrate side inside the photo-curing resin, and a cross section of the first region substantially perpendicular to the extending direction of the first region has a triangle shape; and
the second region extends to the substrate side inside the photo-curing resin, and
at the fourth step, a horizontal hole extending along the substrate is formed like a tunnel having a triangular sectional shape by removing the first region of the non-transmitting region.

27. The flow path forming method according to claim 26, wherein
at the first step, the light shielding pattern is formed on the one main surface of the substrate by using a conductive material, and
at the second step, the one main surface of the substrate is coated with the photo-curing resin.

28. A flow path forming method comprising:
a first step of coating one main surface of a transparent substrate with a photo-curing resin;
a second step of arranging a mask member having a light shielding pattern along another main surface of the substrate;
a third step of irradiating the mask member with the light at different angles from the mask member side so as to transmit the light through a region other than a non-transmitting region extending along the light shielding pattern inside the photo-curing resin, and thereby curing a portion that light penetrated through of the photo-curing resin; and
a fourth step of removing the non-transmitting region of the photo-curing resin, wherein
at the first step, the light shielding pattern includes a first portion having relatively long length in an extending direction of the first portion and relatively short width in a direction substantially perpendicular to the extending direction of the first portion,
at the third step:
the non-transmitting region includes a first region corresponding to the first portion of the light shielding pattern;
only one of the first region of the non-transmitting region is formed corresponding to one of the first portion of the light shielding pattern; and
the first region extends to the substrate side inside the photo-curing resin, and a cross section of the first region substantially perpendicular to the extending direction of the first region has a triangle shape, and
at the fourth step, a horizontal hole extending along the substrate is formed like a tunnel having a triangular sectional shape by removing the first region of the non-transmitting region.

29. The cell clamping device of claim 21, the communication hole has a triangular sectional shape substantially perpendicular to an extending direction of the communication hole toward the pore, one side of which bounds on the conductive film.

30. The cell clamping device of claim 22, the communication hole has a triangular sectional shape substantially perpendicular to an extending direction of the communication hole toward the pore, one side of which bounds on the conductive film.
